(19) United States
(12) Patent Application Publication
(10) Pub. No.: US 2016/0122354 A1
(43) Pub. Date: May 5, 2016

(54) PROCESS FOR THE PREPARATION OF (3R,4R)-4-METHYL-3-(METHYL-7H-PYRROLO[2,3-D]PYRIMIDIN-4-YL-AMINO)-ß-OXO-1-PIPERIDINEPROPANENITRILE AND ITS SALTS

(71) Applicants: Srinivasan THIRUMALAI RAJAN, Andhra Pradesh Hyderabad (IN); Sajja ESWARAIAH, Andhra Pradesh Hyderabad (IN); Revu SATYANARAYANA, Andhra Pradesh Hyderabad (IN); MSN LABORATORIES LIMITED, Andhra Pradesh Hyderabad (IN)

(72) Inventors: Srinivasan THIRUMALAI RAJAN, Hyderabad, Andhra Pradesh (IN); Sajja ESWARAIAH, Hyderabad, Andhra Pradesh (IN); Revu SATYANARAYANA, Hyderabad, Andhra Pradesh (IN)

(21) Appl. No.: 14/895,707

(22) PCT Filed: Jun. 4, 2014
(86) PCT No.: PCT/IN2014/000382

§ 371 Date: Dec. 3, 2015

(30) Foreign Application Priority Data
Jun. 5, 2013 (IN) 2454/CHE/2013

(51) Int. Cl. C07D 487/04; C07C 59/265

(52) U.S. Cl. CPC C07D 487/04; C07C 59/265

(57) ABSTRACT

The present invention relates to an improved 7H-pyrrolo[2,3-d]pyrimidin-4-yl-amino)-β-oxo-1-piperidine propanenitrile compound of formula-1 and its pharmaceutically acceptable salts.

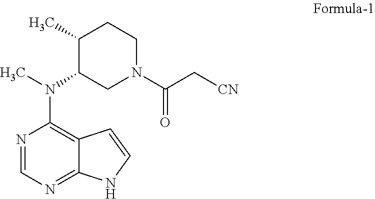 [SMILES: C[C@@H]1CCN(C(=O)CC#N)C[C@@H]1N(C)c1ncnc2[nH]ccc12]

### RELATED APPLICATION

This application claims the benefit of priority of our Indian patent application 2454/CHE/2013 filed on 5 Jun. 2013 which is incorporated herein by reference.

### FIELD OF THE INVENTION

The present invention provides an improved process for the preparation of (3R,4R)-4-methyl-3-(methyl-7H-pyrrolo[2,3-d]pyrimidin-4-ylamino)-β-oxo-1-piperidine propanenitrile compound of formula-1 and its pharmaceutically acceptable salts.

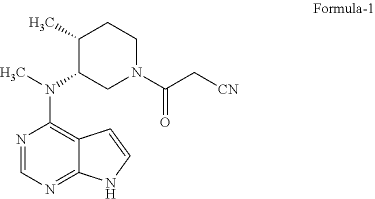 [SMILES: C[C@@H]1CCN(C(=O)CC#N)C[C@@H]1N(C)c1ncnc2[nH]ccc12]

(3R,4R)-4-methyl-3-(methyl-7H-pyrrolo[2,3-d]pyrimidin-4-ylamino)-β-oxo-1-piperidine propanenitrile is a drug discovered and developed by Pfizer. It is currently approved for the treatment of rheumatoid arthritis (RA) in the United States and is being studied for the treatment of psoriasis, inflammatory bowel disease and other immunological diseases, as well as for the prevention of organ transplant rejection.

### BACKGROUND OF THE INVENTION

The (3R,4R)-4-methyl-3-(methyl-7H-pyrrolo[2,3-d]pyrimidin-4-ylamino)-β-oxo-1-piperidine propanenitrile is commonly known as Tofacitinib. The synthesis of (3R,4R)-4-methyl-3-(methyl-7H-pyrrolo[2,3-d]pyrimidin-4-ylamino)-β-oxo-1-piperidine propanenitrile and its intermediates have previously been described in U.S. Pat. No. 6,627,754, U.S. Pat. No. 7,301,023, U.S. Pat. No. 6,965,027 and U.S. Pat. No. 7,084,277.

U.S. Pat. No. 6,627,754 describes the compound 3-{4-methyl-(7H-pyrrolo[2,3-d]pyrimidin-4-yl)-amino}-piperidin-1-yl)-3-oxo-propionitrile and its pharmaceutically acceptable salts, which are useful inhibitors of protein kinases (such as the enzyme JAK 3) and as such are useful for therapy as immunosuppressive agents for organ transplants, xeno transplantation, lupus, multiple sclerosis, rheumatoid arthritis, psoriasis, Type 1 diabetes and complication from diabetes, cancer, atopic dermatitis, autoimmune thyroid disorders, ulcerative colitis, Crohn's disease, Alzheimer's disease, leukemia and other indications where immune suppression would be desirable.

The preparation of (3R,4R)-4-methyl-3-(methyl-7H-pyrrolo[2,3-d]pyrimidin-4-ylamino)-β-oxo-1-piperidine propanenitrile mono citrate salt was described in U.S. Pat. No. 6,965,027.

U.S. Pat. No. U.S. Pat. No. 7,084,277 describes the synthesis of an intermediate, cis-(1-benzyl-4-methyl-piperidin-3-yl)-methyl-amine hydrochloride salt, which is useful in the synthesis of (3R,4R)-4-methyl-3-(methyl-7H-pyrrolo[2,3-d]pyrimidin-4-ylamino)-β-oxo-1-piperidine propanenitrile and its corresponding citrate salt.

Still, there is a need in the art to develop an alternate process for the synthesis of (3R,4R)-4-methyl-3-(methyl-7H-pyrrolo[2,3-d]pyrimidin-4-ylamino)-β-oxo-1-piperidine propanenitrile and its salts which enhances the yield as well as decreases the reaction time.

### BRIEF DESCRIPTION OF THE INVENTION

The first aspect of the present invention is to provide a process for the preparation of (3R,4R)-4-methyl-3-(methyl-7H-prrolo[2,3-d]pyrimidin-4-ylamino)-β-oxo-1-piperidine propanenitrile compound of formula-1 and its acid-addition salts, comprising of;
a) Reacting the (3R,4R)-1-benzyl-N,4-dimethylpiperidin-3-amine compound of formula-2 with compound of general formula-3 in presence of a suitable base in a suitable solvent to provide compound of general formula-4,
b) treating the compound of general formula-4 with a suitable debenzylating agent in a suitable solvent to provide compound of general formula-5,
c) deprotecting the compound of general formula-5 by treating it with a suitable deprotecting agent in a suitable solvent to provide N-methyl-N-((3R,4R)-4-methylpiperidin-3-yl)-7H-pyrrolo[2,3-d]pyrimidin-4-amine compound of formula-6,
d) reacting the compound of formula-6 with 2-cyanoacetyl derivative compound of general formula-(a) in presence of a suitable base in a suitable solvent to provide compound of formula-1,
e) converting the compound of formula-1 into its acid-addition salt by treating it with a suitable acid in a suitable solvent.

The second aspect of the present invention is to provide a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of
a) Reacting the 1-benzyl-N,4-dimethylpiperidin-3-amine compound of formula-7 with 4-chloro-7H-pyrrolo[2,3-d]pyrimidine compound of formula-8 in presence of a suitable base in a suitable solvent to provide N-(1-benzyl-4-methylpiperidin-3-yl)-N-methyl-7H-pyrrolo[2,3-d]pyrimidin-4-amine compound of formula-9,
b) treating the compound of formula-9 with a suitable debenzylating agent in a suitable solvent to provide N-methyl-N-(4-methylpiperidin-3-yl)-7H-pyrrolo[2,3-d]pyrimidin-4-amine compound of formula-10,
c) treating the compound of formula-10 with a suitable chiral acid in a suitable solvent to provide N-methyl-N-((3R,4R)-4-methylpiperidin-3-yl)-7H-pyrrolo[2,3-d]pyrimidin-4-amine compound of formula-6,
d) reacting the compound of formula-6 with 2-cyanoacetyl derivative compound of general formula-(a) in presence of a suitable base in a suitable solvent to provide compound of formula-1.

The third aspect of the present invention is to provide a process for the preparation of N-methyl-N-((3R,4R)-4-methylpiperidin-3-yl)-7H-pyrrolo[2,3-d]pyrimidin-4-amine compound of formula-6, comprising of treating the N-methyl-N-(4-methylpiperidin-3-yl)-7H-pyrrolo[2,3-d]pyrimidin-4-amine compound of formula-10 with a suitable chiral acid in a suitable solvent to provide compound of formula-6.

The fourth aspect of the present invention is to provide a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of;
a) Reacting the (3R,4R)-1-benzyl-N,4-dimethylpiperidin-3-amine compound of formula-2 with a suitable protecting agent in a suitable solvent to provide compound of general formula-11,
b) treating the compound of general formula-11 with a suitable debenzylating agent in a suitable solvent to provide compound of general formula-12,
c) reacting the compound of general formula-12 with 2-cyanoacetyl derivative compound of general formula-(a) in presence of a suitable base in a suitable solvent to provide compound of general formula-13,
d) deprotecting the compound of general formula-13 by treating it with a suitable deprotecting agent in a suitable solvent to provide 3-((3R,4R)-4-methyl-3-(methylamino)piperidin-1-yl)-3-oxo propanenitrile compound of formula-14,
e) reacting the compound of formula-14 with 4-chloro-7H-pyrrolo[2,3-d]pyrimidine compound of formula-8 in presence of a suitable base in a suitable solvent to provide compound of formula-1.

The fifth aspect of the present invention is to provide a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of
a) Reacting the (3R,4R)—N,4-dimethylpiperidin-3-amine compound of formula-15 with a suitable protecting agent in a suitable solvent to provide compound of general formula-12,
b) reacting the compound of general formula-12 with substituted benzyl halide compound of general formula-16 in presence of a base in a suitable solvent to provide compound of general formula-17,
c) treating the compound of general formula-17 with a suitable deprotecting agent in a suitable solvent to provide compound of general formula-18,
d) reacting the compound of general formula-18 with compound of general formula-3 in presence of a suitable base in a suitable solvent to provide compound of general formula-19,
e) treating the compound of general formula-19 with a suitable deprotecting agent in a suitable solvent to provide N-methyl-N-((3R,4R)-4-methylpiperidin-3-yl)-7H-pyrrolo[2,3-d]pyrimidin-4-amine compound of formula-6,
f) reacting the compound of formula-6 with 2-cyanoacetyl derivative compound of general formula-(a) in presence of a suitable base in a suitable solvent to provide compound of formula-1.

The sixth aspect of the present invention is to provide novel intermediate compound which is useful for the preparation of (3R,4R)-4-methyl-3-(methyl-7H-pyrrolo[2,3-d]pyrimidin-4-ylamino)-β-oxo-1-piperidine propanenitrile compound of formula-1.

### DETAILED DESCRIPTION OF THE INVENTION

The term “suitable solvent” used in the present invention refers to “hydrocarbon solvents” such as n-hexane, n-heptane, cyclohexane, petroleum ether, benzene, toluene, xylene and the like; “ether solvents” such as dimethyl ether, diethyl ether, diisopropyl ether, methyl tert-butyl ether, 1,2-dimethoxy ethane, tetrahydrofuran, 1,4-dioxane and the like; “ester solvents” such as methyl acetate, ethyl acetate, n-propyl acetate, isopropyl acetate, n-butyl acetate, isobutyl acetate, tert-butyl acetate and the like; “polar-aprotic solvents” such as dimethylacetamide, dimethylformamide, dimethylsulfoxide, N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcohol solvents” such as methanol, ethanol, n-propanol, iso-propanol, n-butanol, iso-butanol, tert-butanol, ethane-1,2-diol, propane-1,2-diol and the like; “polar solvents” such as water; acetic acid, formic acid or their mixtures.

The term “suitable base” used in the present invention refers to inorganic bases selected from “alkali metal carbonates” such as sodium carbonate, potassium carbonate, lithium carbonate, cesium carbonate and the like; “alkali metal bicarbonates” such as sodium bicarbonate, potassium bicarbonate, lithium bicarbonate, cesium bicarbonate and the like; “alkali metal hydroxides” such as sodium hydroxide, potassium hydroxide, lithium hydroxide and the like; “alkali metal alkoxides” such as sodium methoxide, sodium ethoxide, potassium methoxide, potassium ethoxide, sodium tert.butoxide, potassium tert.butoxide, lithium tert.butoxide and the like; alkali metal hydrides such as sodium hyd ride, potassium hydride, lithium hydride and the like; alkali metal amides such as sodium amide, potassium amide, lithium amide and the like; ammonia, alkali metal and alkaline earth metal salts of acetic acid such as sodium acetate, potassium acetate, magnesium acetate, calcium acetate and the like; “organic bases” like dimethylamine, diethylamine, diisopropyl amine, isopropyl ethylamine, diisopropylethylamine, di n-butylamine, diisobutylamine, triethylamine, tributylamine, tert-butyl amine, pyridine, piperidine, 4-dimethylaminopyridine (DMAP), 1,8-diazabicyclo[5.4.0]undec-7-ene (DBU), 1,5-diazabicyclo[4.3.0]non-5-ene (DBN), N-methylmorpholine (NMM), 1,4-diazabicyclo[2.2.2]octane (DABCO), 2,6-lutidine, lithium diisopropylamide; “organolithium bases” such as n-butyl lithium, “organosilicon bases” such as lithium hexamethyldisilazide (LiHMDS), sodium hexamethyldisilazide (NaHMDS), potassium hexamethyldisilazide (KHMDS) or their mixtures.

As used herein the present invention the term “chiral acid” refers to mandelic acid, acetyl mandelic acid, tartaric acid, di-p-tolyl tartaric acid, dibenzoyl tartaric acid, di-p-anisoyl tartaric acid, camphor sulfonic acid and the like.

Throughout this document, “PG” represents N-protecting group selected from but not limited to acetyl, trifluoroacetyl, aralkyl wherein aryl is optionally substituted with one or more substituents like C₁-C₆ alkyl, alkoxy, nitro halo and the like such as substituted or unsubstituted benzyl, p-methoxybenzyl (PMB) and the like; benzoyl, benzyloxy carbonyl, tert.butyloxy carbonyl, substituted or unsubstituted alkyl/aryl sulfonyl; fluorenylmethyloxycarbonyl (Fmoc), tri(C₁-C₆ straight chain or branched chain)alkyl silyl groups such as trimethyl silyl, triethyl silyl, triisopropylsilyl, tert.butyl dimethylsilyl; trityl, 4-methyl trityl, 4,4-dimethoxy trityl, 3,5-dimethoxy phenylisopropoxy carbonyl, 2-[4-biphenyl]isopropoxy carbonyl, nitrophenyl sulphonyl, 2-(4-nitrophenylsulfonyl)ethoxy carbonyl, 2-(methylsulfonyl)ethoxycarbonyl, phenylhydrazine. The said N-protecting group can be removed by treating the corresponding N-protected compound with a suitable acid or a suitable base or by hydrogenolysis depending on the nature of the protecting group employed.

The term “protecting agent” refers to acetic acid, acetyl chloride, acetic anhydride, trifluoroacetic acid, trifluoroacetyl chloride, trifluoroacetic anhydride, substituted or unsubstituted aralkyl halides, benzoyl halides, benzoic anhydride, benzyl chloroformate, di-tert.butyl dicarbonate (DIBOC), substituted or unsubstituted alkyl/aryl sulfonic acids/acid halides/anhydrides, fluorenylmethyloxy carbonyl chloride (Fmoc chloride), tri(C₁-C₆ straight chain or branched chain)alkyl silyl halides, trityl chloride, 4-methyl trityl chloride, 4,4-dimethoxy trityl chloride and the like.

In the present invention the suitable deprotecting agent is selected based on the protecting group employed. The suitable deprotecting agent is selected from but not limited to acids such as hydrochloric acid, hydrobromic acid, sulfuric acid, nitric acid, aq.phosphoric acid, trifluoroacetic acid, methane sulfonic acid; acetyl chloride in combination with alcohols; bases such as alkali metal hydroxides, alkali metal carbonates, cesium carbonate/imidazole, alkali metal bicarbonates, ammonia; and organic bases such as methylamine, ethylamine, diethylamine, triethylamine, piperidine and the like; hydrogenating agents such as Pd/C, Pd(OH)₂/C (Pearlman's catalyst), palladium acetate, platinum oxide, platinum black, sodium borohydride, Na-liquid ammonia, Raney-Ni, tri(C₁-C₆)alkylsilanes, tri(C₁-C₆)alkylsilyl halides and the like.

The first aspect of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1 and its acid-addition salts, comprising of;
a) Reacting the (3R,4R)-1-benzyl-N,4-dimethylpiperidin-3-amine compound of formula-2

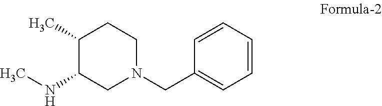 [SMILES: CN[C@H]1CN(Cc2ccccc2)CC[C@H]1C]

with compound of general formula-3

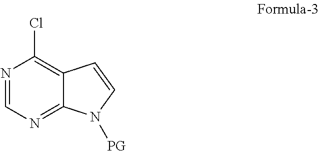

wherein, ‘PG’ represents N-protecting group as defined above;
in presence of a suitable base in a suitable solvent to provide compound of general formula-4,

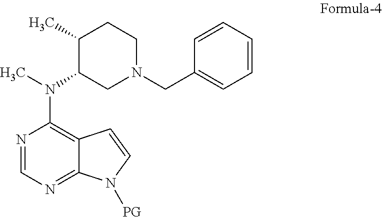

b) treating the compound of general formula-4 with a suitable debenzylating agent in a suitable solvent to provide compound of general formula-5,

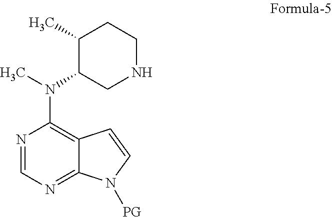

c) deprotecting the compound of general formula-5 by treating it with a suitable deprotecting agent in a suitable solvent to provide N-methyl-N-((3R,4R)-4-methylpiperidin-3-yl)-7H-pyrrolo[2,3-d]pyrimidin-4-amine compound of formula-6,

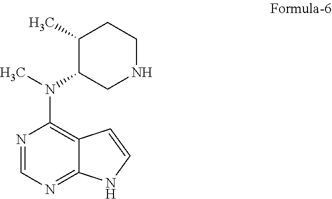

d) reacting the compound of formula-6 with 2-cyanoacetyl derivative compound of general formula-(a) in presence of a suitable base in a suitable solvent to provide compound of formula-1,
e) converting the compound of formula-1 into its acid-addition salt by treating it with a suitable acid in a suitable solvent.

Wherein, in step-(a) & step-(d) the suitable base is selected from organic bases, inorganic bases or their mixtures;

In step-(b) the suitable debenzylating agent is selected from conc. HCl, Pd, Pd/C, Pd(OH)₂/C, palladium acetate, Raney Ni, Pt/C, platinum oxide, platinum black, Rh/C, Ru, Ir and the like optionally in combination with hydrogen;

In step-(c) the suitable deprotecting agent is selected from acids, bases, hydrogenating agents based on the protecting group employed;

In step-e) the suitable acid is preferably citric acid (2-hydroxy-1,2,3-propanetricarboxylic acid);

in step-(a) to step-(e) the suitable solvent is selected from ether solvents, ester solvents, chloro solvents, hydrocarbon solvents, polar solvents, polar-aprotic solvents, ketone solvents, alcohol solvents, nitrile solvents, acetic acid, formic acid or their mixtures.

The compound of formula-2 can be optionally purified by converting it into its acid, addition salts and optionally slurring in the suitable solvent to get the compound of formula-2 having less than 0.1% of diastereomer impurities.

The compound of formula-2 is converted into its hydrochloride salt and slurrying in ethanol to provide highly pure compound of formula-2 with less than 0.1% of diastereomer impurities.

Alternatively, the compound of formula-1 can also be prepared by reacting the compound of general formula-5 with 2-cyanoacetyl derivative compound of general formula-(a) in presence of a suitable base in a suitable solvent followed by deprotection with a suitable deprotecting agent in a suitable solvent.

Another aspect of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1 and its citrate salt compound of formula-1a, comprising of;
a) Reacting the 4-chloro-7H-pyrrolo[2,3-d]pyrimidine compound of formula-8 with methanesulphonyl chloride in presence of a suitable base in a suitable solvent to provide 4-chloro-7-(methylsulfonyl)-7H-pyrrolo[2,3-d]pyrimidine compound of formula-3a,

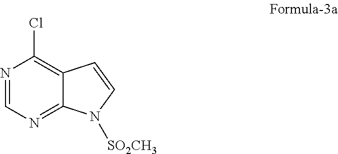 [SMILES: CS(=O)(=O)n1ccc2c(Cl)ncnc21]

b) reacting the compound of formula-3a with (3R,4R)-1-benzyl-N,4-dimethylpiperidin-3-amine compound of formula-2 in presence of a suitable base in a suitable solvent to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a,

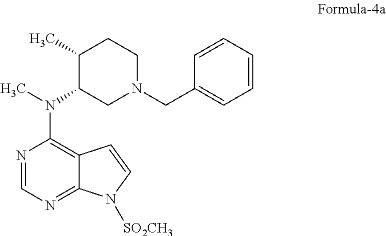 [SMILES: C[C@@H]1CCN(Cc2ccccc2)C[C@@H]1N(C)c1ncnc2c1ccn2S(C)(=O)=O]

c) deprotecting the compound of formula-4a by treating it with a suitable base in a suitable solvent to provide N-((3R,4R)-1-benzyl-4-methylpiperidin-3-yl)-N-methyl-7H-pyrrolo[2,3-d]pyrimidin-4-amine compound of formula-21,

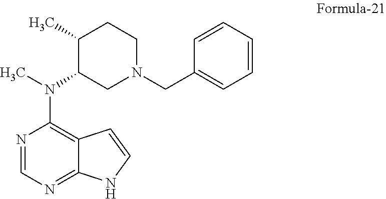 [SMILES: C[C@@H]1CCN(Cc2ccccc2)C[C@@H]1N(C)c1ncnc2[nH]ccc12]

d) treating the compound of formula-21 with a suitable debenzylating agent in a suitable solvent to provide N-methyl-N-((3R,4R)-4-methylpiperidin-3-yl)-7H-pyrrolo[2,3-d]pyrimidin-4-amine compound of formula-6,
e) reacting the compound of formula-6 with 2-cyanoacetyl derivative compound of general formula-(a) in presence of a suitable base in a suitable solvent to provide compound of formula-1,
f) treating the compound of formula-1 with citric acid in a suitable solvent to provide citrate salt compound of formula-1.

Wherein, in step-(a), step-(b) & step-(e) the suitable base is same as defined in step-(a) of the first aspect of the present invention;

In step-(c) the suitable base is selected from alkali metal hydroxides, alkali metal alkoxides;

In step-(d) the suitable debenzylating agent is same as defined in step-(b) of the first aspect of the present invention;

In step-(a) to step-(f) the suitable solvent is same as defined in step-(a) of the first aspect of the present invention.

Debenzylation of compound of formula-4a at higher temperature leads to the formation of corresponding dihydro impurity which in the further stages is converted into dihydro Tofacitinib present as an impurity in the final product beyond the ICH limits.

In the present invention, the debenzylation was carried out at room temperature (25-30° C.) which controls the formation of dihydro impurity, and further dihydro Tofacitinib to the limits of NMT 0.15% in the final API.

A preferred embodiment of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1 and its citrate salt compound of formula-1a, comprising of;
a) Reacting the 4-chloro-7H-pyrrolo[2,3-d]pyrimidine compound of formula-8 with methanesulphonyl chloride in presence of triethyl amine in acetone to provide 4-chloro-7-(methylsulfonyl)-7H-pyrrolo[2,3-d]pyrimidine compound of formula-3a,
b) reacting the compound of formula-3a with (3R,4R)-1-benzyl-N,4-dimethylpiperidin-3-amine compound of formula-2 in presence of sodium bicarbonate in acetonitrile to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a,
c) deprotecting the compound of formula-4a by treating it with aqueous sodium hydroxide in toluene to provide N-((3R,4R)-1-benzyl-4-methylpiperidin-3-yl)-N-methyl-7H-pyrrolo[2,3-d]pyrimidin-4-amine compound of formula-21,
d) treating the compound of formula-21 with palladium hydroxide in aqueous isopropyl alcohol and in presence of catalytic amount of acetic acid to provide N-methyl-N-((3R,4R)-4-methylpiperidin-3-yl)-7H-pyrrolo[2,3-d]pyrimidin-4-amine compound of formula-6,
e) reacting the compound of formula-6 with ethyl cyanoacetate compound of formula-al

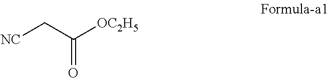

in presence of 1,8-diazabicyclo[5.4.0]undec-7-ene in n-butanol to provide compound of formula-1,
f) treating the compound of formula-1 with citric acid in aqueous n-butanol to provide compound of formula-1a.

Another aspect of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of;
a) Reacting the 4-chloro-7H-pyrrolo[2,3-d]pyrimidine compound of formula-8 with methanesulphonyl chloride in presence of a suitable base in a suitable solvent to, provide 4-chloro-7-(methylsulfonyl)-7H-pyrrolo[2,3-d]pyrimidine compound of formula-3a,
b) reacting the compound of formula-3a with (3R,4R)-1-benzyl-N,4-dimethylpiperidin-3-amine compound of formula-2 in presence of a suitable base in a suitable solvent to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a,
c) treating the compound of formula-4a with a suitable debenzylating agent in a suitable solvent to provide N-methyl-N-((3R,4R)-4-methylpiperidin-3-yl)-7-(methylsulfonyl)-7H-pyrrolo[2,3-d]pyrimidin-4-amine compound of formula-5a,

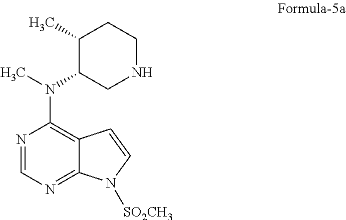 [SMILES: C[C@@H]1CCNC[C@@H]1N(C)c1ncnc2c1ccn2S(C)(=O)=O]

d) deprotecting the compound of formula-5a by treating it with a suitable base in a suitable solvent to provide N-methyl-N-((3R,4R)-4-methylpiperidin-3-yl)-7H-pyrrolo[2,3-d]pyrimidin-4-amine compound of formula-6,
e) reacting the compound of formula-6 with 2-cyanoacetyl derivative compound of general formula-(a) in presence of a suitable base in a suitable solvent to provide compound of formula-1,
f) treating the compound of formula-1 with citric acid in a suitable solvent to provide citrate salt compound of formula-1.

Wherein, in step-(a), step-(b) & step-(e) the suitable base is same as defined in step-(a) of the first aspect of the present invention;

In step-(c) the suitable debenzylating agent is same as defined in step-(b) of the first aspect of the present invention;

In step-(d) the suitable base is selected from alkali metal hydroxides and alkali metal alkoxides;

In step-(a) to step-(f) the suitable solvent is same as defined in step-(a) of the first aspect of the present invention.

Alternatively, the compound of formula-1 can also be prepared by reacting the compound of general formula-5a with 2-cyanoacetyl derivative compound of general formula-(a) in presence of a suitable base in a suitable solvent followed by deprotection with a suitable base in a suitable solvent.

The second aspect of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of
a) Reacting the 1-benzyl-N,4-dimethylpiperidin-3-amine compound of formula-7

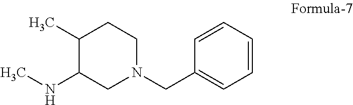 [SMILES: CNC1CN(Cc2ccccc2)CCC1C]

with 4-chloro-7H-pyrrolo[2,3-d]pyrimidine compound of formula-8

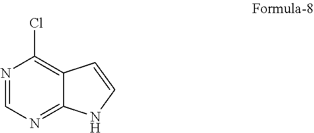

in presence of a suitable base in a suitable solvent to provide N-(1-benzyl-4-methylpiperidin-3-yl)-N-methyl-7H-pyrrolo[2,3-d]pyrimidin-4-amine compound of formula-9,

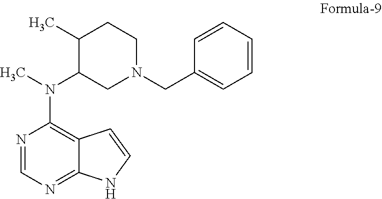 [SMILES: CC1CCN(Cc2ccccc2)CC1N(C)c1ncnc2[nH]ccc12]

b) treating the compound of formula-9 with a suitable debenzylating agent in a suitable solvent to provide N-methyl-N-(4-methylpiperidin-3-yl)-7H-pyrrolo[2,3-d]pyrimidin-4-amine compound of formula-10,

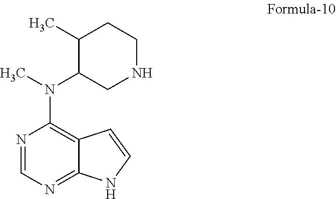 [SMILES: CC1CCNCC1N(C)c1ncnc2[nH]ccc12]

c) treating the compound of formula-10 with a suitable chiral acid in a suitable solvent to provide N-methyl-N-((3R,4R)-4-methylpiperidin-3-yl)-7H-pyrrolo[2,3-d]pyrimidin-4-amine compound of formula-6,
d) reacting the compound of formula-6 with 2-cyanoacetyl derivative compound of general formula-(a) in presence of a suitable base in a suitable solvent to provide compound of formula-1.

Wherein in step-(a) and step-(d) the suitable base and the suitable solvent are same as defined in step-(a) of the first aspect of the present invention;

In step-(b) the suitable debenzylating agent is same as defined in step-(b) of the first aspect of the present invention;

In step-(c) the suitable chiral acid is selected from but not limited to mandelic acid, acetyl mandelic acid, tartaric acid, di-p-tolyl tartaric acid, dibenzoyl tartaric acid, di-p-anisoyl tartaric acid, camphor sulfonic acid and the like;

In step-(b) and step-(c) the suitable solvent is same as defined in step-(a) of the first aspect of the present invention.

The third aspect of the present invention provides a process for the preparation of N-methyl-N-((3R,4R)-4-methylpiperidin-3-yl)-7H-pyrrolo[2,3-d]pyrimidin-4-amine compound of formula-6, comprising of treating the N-methyl-N-(4-methylpiperidin-3-yl)-7H-pyrrolo[2,3-d]pyrimidin-4-amine compound of formula-10 with a suitable chiral acid in a suitable solvent to provide compound of formula-6.

Wherein, the suitable chiral acid and the suitable solvent are same as defined in step-c) of the second aspect of the present invention.

The fourth aspect of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of;
a) Reacting the (3R,4R)-1-benzyl-N,4-dimethylpiperidin-3-amine compound of formula-2 with a suitable protecting agent in a suitable solvent to provide compound of general

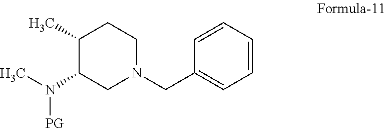

wherein, ‘PG’ represents N-protecting group as defined above;
b) treating the compound of general formula-11 with a suitable debenzylating agent in a suitable solvent to provide compound of general formula-12,

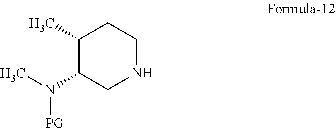

c) reacting the compound of general formula-12 with 2-cyanoacetyl derivative compound of general formula-(a) in presence of a suitable base in a suitable solvent to provide compound of general formula-13,

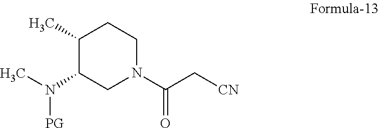

d) deprotecting the compound of general formula-13 by treating it with a suitable deprotecting agent in a suitable solvent to provide 3-((3R,4R)-4-methyl-3-(methylamino)piperidin-1-yl)-3-oxopropane nitrile compound of formula-14,

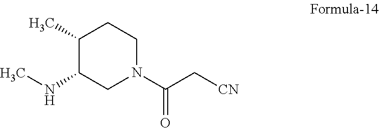

e) reacting the compound of formula-14 with 4-chloro-7H-pyrrolo[2,3-d]pyrimidine compound of formula-8 in presence of a suitable base in a suitable solvent to provide compound of formula-1.

Wherein, in step-a) the suitable protecting agent is same as defined above;

In step-(b), the suitable debenzylating agent is same as defined in step-(b) of the first aspect of the present invention;

In step-(d) the suitable deprotecting agent is same as defined in step-(c) of the first aspect of the present invention;

In step-(c) & step-(e) the suitable base is same as defined in step-(a) of the first aspect of the present invention;

In step-(a) to step-(e) the suitable solvent is same as defined in step-(a) of the first aspect of the present invention.

The fifth aspect of the present invention provides a process for the preparation of (3R,4R)-4-methyl-3-(methyl-7H-pyrrolo[2,3-d]pyrimidin-4-ylamino)-β-oxo-1-piperidine propanenitrile compound of formula-1, comprising of;
a) Reacting the (3R,4R)—N,4-dimethylpiperidin-3-amine compound of formula-15

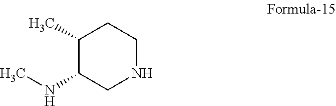

with a suitable protecting agent in a suitable solvent to provide compound of general formula-12,
b) reacting the compound of general formula-12 with aralkyl halide compound of general formula-16

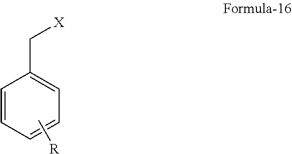

wherein, X=Cl or Br; R=alkyl, alkoxy, halo or cyano;
in presence of a base in a suitable solvent to provide compound of general formula-17,

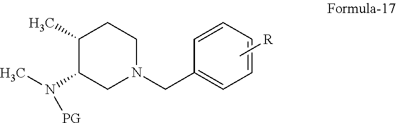

wherein, ‘PG’ represents N-protecting group as defined above;
c) deprotecting the compound of general formula-17 by treating it with a suitable deprotecting agent in a suitable solvent to provide compound of general formula-18,

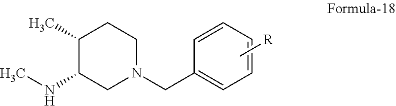

d) reacting the compound of general formula-18 with compound of general formula-3 in presence of a suitable base in a suitable solvent to provide compound of general formula-19,

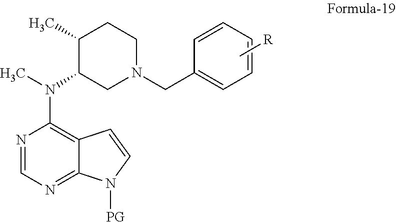

e) treating the compound of general formula-19 with a suitable debenzylating agent in a suitable solvent, optionally further treating with a suitable deprotecting agent in a suitable solvent to provide N-methyl-N-((3R,4R)-4-methylpiperidin-3-yl)-7H-pyrrolo[2,3-d]pyrimidin-4-amine compound of formula-6,
f) reacting the compound of formula-6 with 2-cyanoacetyl derivative compound of general formula-(a) in presence of suitable base in suitable solvent to provide compound of formula-1.

Wherein, in step-(a) the suitable protecting agent is same as defined in step-(a) of the fourth aspect of the present invention;

In step-(b), step-(d) & step-(f) the suitable base is same as defined in step-(a) of the first aspect of the present invention;

In step-(c) & step-(e) the suitable deprotecting agent is same as defined in step-(c) of the first aspect of the present invention;

In step-(e) the suitable debenzylating agent is same as defined in step-(b) of the first aspect of the present invention;

In step-(a) to step-(f) the suitable solvent is same as defined in step-(a) of the first aspect of the present invention.

The sixth aspect of the present invention provides novel intermediate compound which is useful for the preparation of (3R,4R)-4-methyl-3-(methyl-7H-pyrrolo[2,3-d]pyrimidin-4-ylamino)-β-oxo-1-piperidine propanenitrile compound of formula-1. The said novel intermediate compound is represented by the below mentioned structural formula;

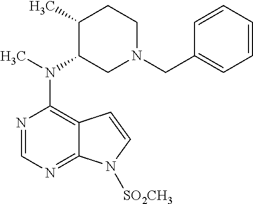 [SMILES: C[C@@H]1CCN(Cc2ccccc2)C[C@@H]1N(C)c1ncnc2c1ccn2S(C)(=O)=O]

### HPLC Method of Analysis of Tofacitinib Citrate:

Apparatus: A liquid chromatographic system equipped with variable wavelength UV-detector; Column: Kromasil C18, 250×4.6 mm, 5 μm or equivalent; Flow rate: 1.0 mL/min; Wavelength: 210 nm; Column temperature: 25° C.; Injection volume: 10 μL; Run time: 52 min; Diluent: water:acetonitrile (80:20 v/v); Elution: gradient; Buffer: Weigh accurately 2.72 gm of potassium dihydrogen phosphate and 1 g of 1-octane sulphonic acid sodium salt anhydrous in 1000 ml of milli-Q-water. Adjust the pH to 5.5 with dil.KOH solution and filtered the solution through 0.22 μm filter paper. Mobile phase-A: Buffer:acetonitrile (90:10 v/v); Mobile phase-B: acetonitrile:buffer (70:30 v/v).

The PXRD analysis of compound of formula-1a of the present invention was carried out using BRUKER/AXS X-Ray diffractometer using Cu Kα radiation of wavelength 1.5406 A° and continuous scan speed of 0.03°/min.

Tofacitinib citrate produced by the present invention can be further micronized or milled to get the desired particle size to achieve desired solubility profile based on different forms of pharmaceutical composition requirements. Techniques that may be used for particle size reduction include, but not limited to ball mills, roller and hammer mills and jet mills. Milling or micronization may be performed before drying or after drying of the product.

The present invention is schematically represented as follows.

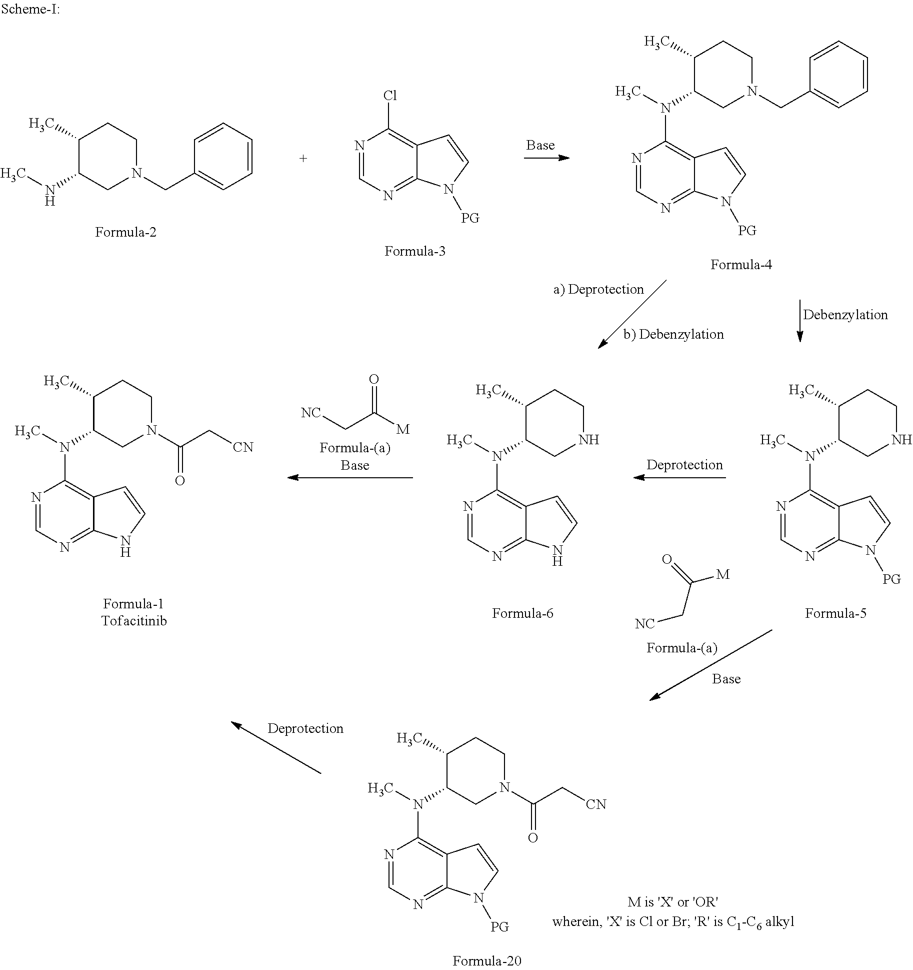

Wherein, “PG” is a “Protecting group” selected from acetyl, trifluoroacetyl, Fmoc, methanesulfonyl, alkylbenzyl, alkoxybenzyl, trimethyl silyl, triethyl silyl, tert.butyl dimethylsilyl, trityl, 4-methyl trityl, and 4,4-dimethoxy trityl.

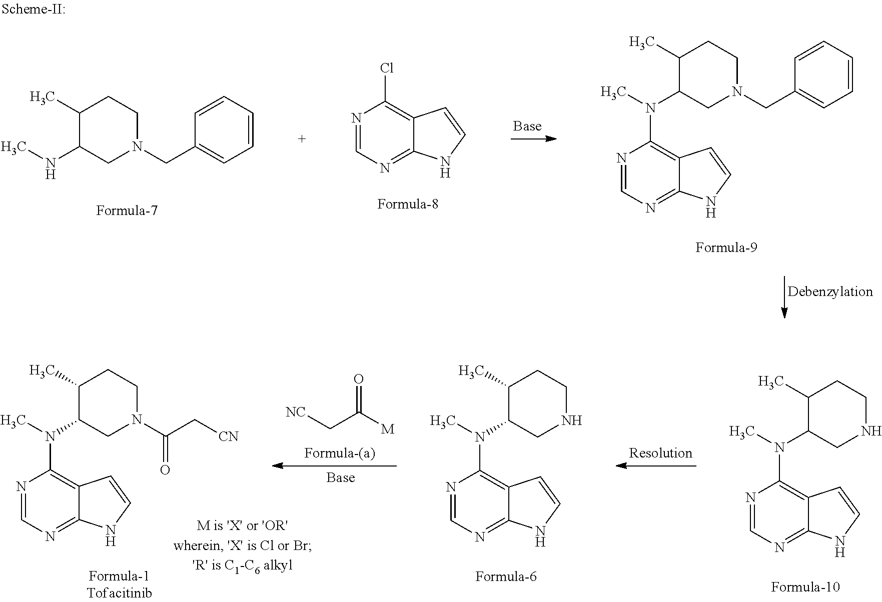

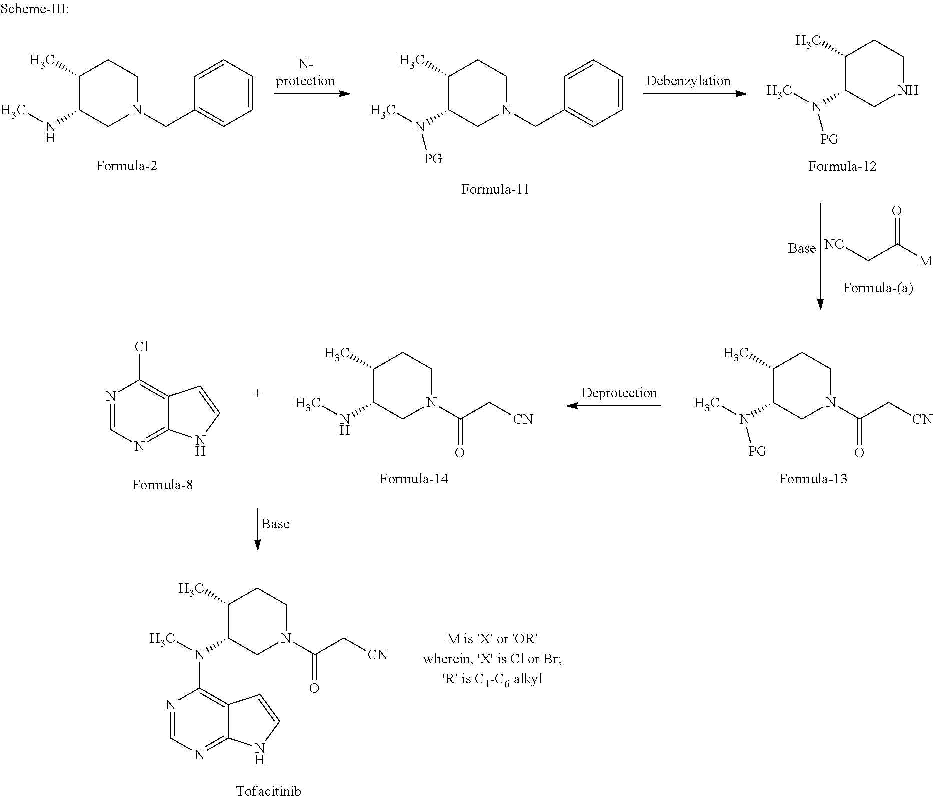

Wherein, “PG” is a “Protecting group” selected from acetyl, trifluoroacetyl, Fmoc, alkyl and aryl sulforlyls wherein, aryl is optionally substituted with alkyl, halogen, cyario and alkoxy; alkylbenzyl, alkoxybenzyl, trimethyl silyl, triethyl silyl, tert.butyl dimethylsilyl,trityl, 4-methyl trityl, and 4,4-dimethoxy trityl.

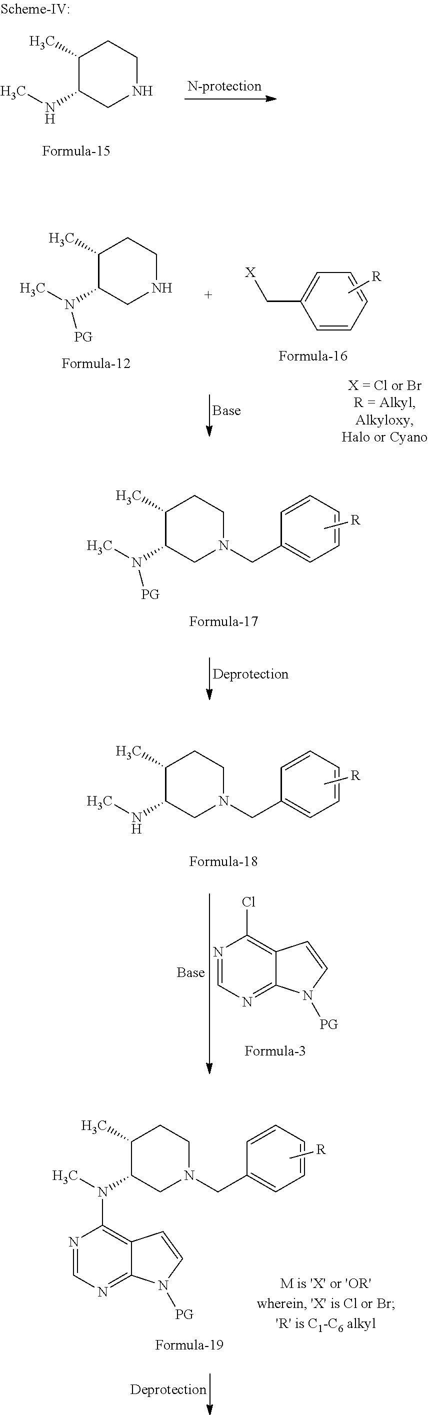

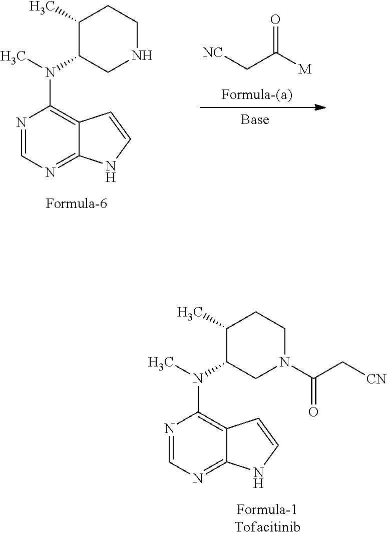

Wherein, “PG” is a “Protecting group” selected from acetyl, trifluoroacetyl, Fmoc, alkyl and aryl sulfonyls wherein, aryl is optionally substituted with alkyl, halogen, cyano and alkoxy; alkylbenzyl, alkoxybenzyl, benzyloxy carbonyl, substituted benzyloxy carbonyl, tertiary butyloxy carbonyl, trimethyl silyl, triethyl silyl, tert.butyl dimethylsilyl, trityl, A-methyl trityl and 4,4-dimethoxy trityl.

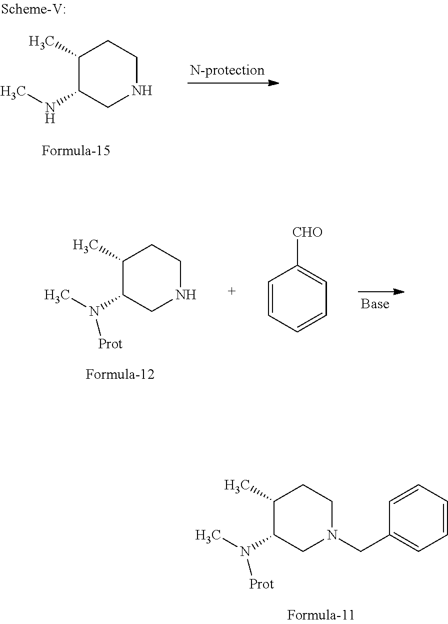

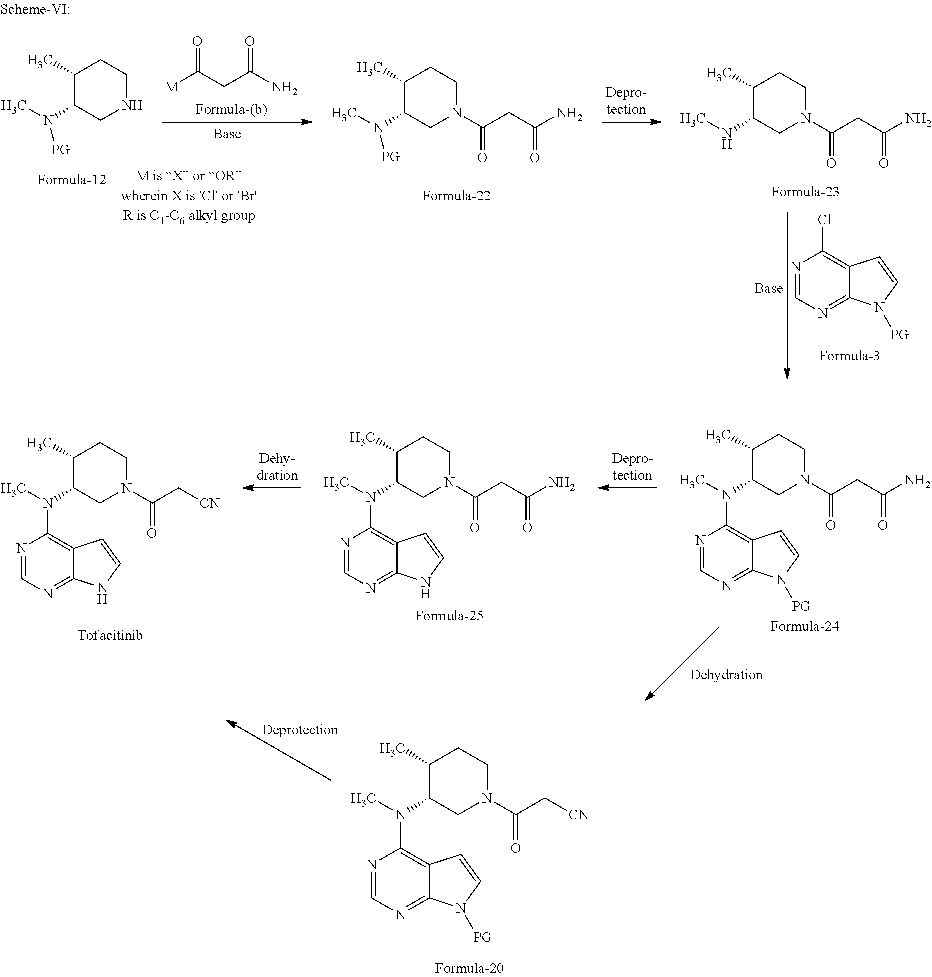

Wherein, “PG” is a “Protecting group” selected from acetyl, trifluoroacetyl, Fmoc, alkyl and aryl sulfonyls wherein, aryl is optionally substituted with alkyl, halogen, cyano and alkoxy; alkylbenzyl, alkoxybenzyl, benzyloxy carbonyl, substituted benzyloxy carbonyl, tertiary butyloxy carbonyl, trimethyl silyl, triethyl silyl, tert.butyl dimethylsilyl, trityl, 4-methyl trityl and 4,4-dimethoxy trityl.

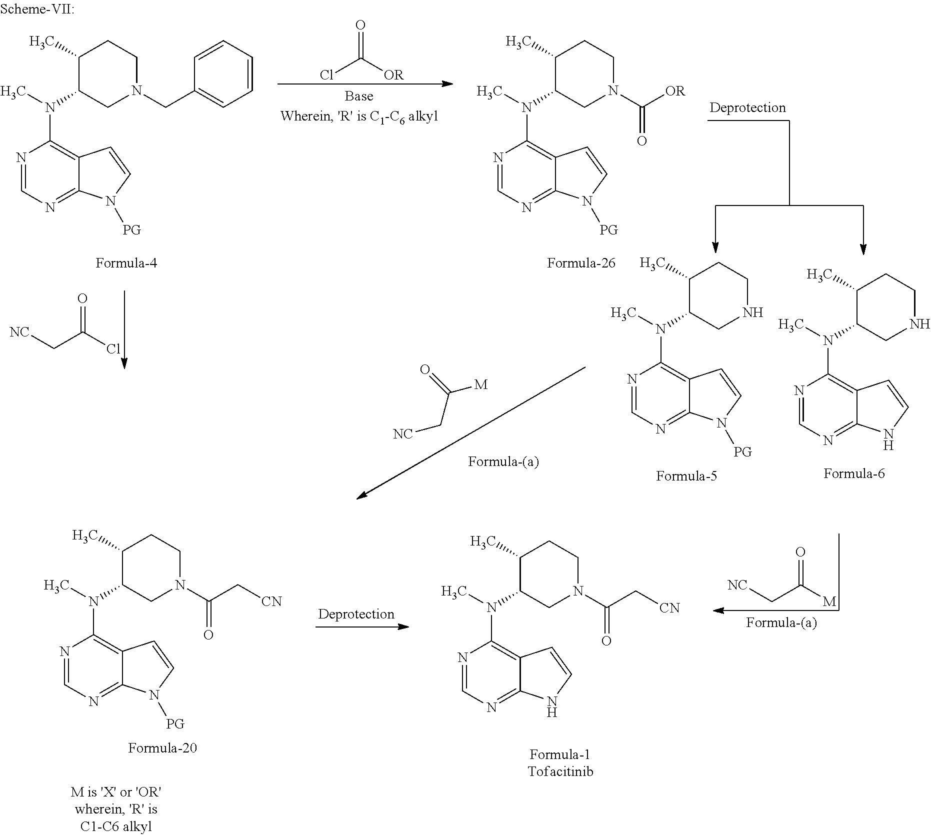

Wherein, “PC” is a “Protecting group” selected from acetyl, trifluoroacetyl, Fmoc, methanesulfonyl, alkylbenzyl, alkoxybenzyl, trimethyl silyl, triethyl silyl, tert.butyl dimethylsilyl, trityl, 4-methyl trityl, and 4,4-dimethoxy trityl.

The process described in the present invention was demonstrated in examples illustrated below. These examples are provided as illustration only and therefore should not be construed as limitation of the scope of the invention.

### EXAMPLES

### Example-1

### Preparation of 4-chloro-7-(methylsulfonyl)-7H-pyrrolo[2,3-d]pyrimidine (Formula-3a)

Methanesulfonyl chloride (111 gm) was added to a mixture of 4-chloro-7H-pyrrolo[2,3-d]pyrimidine compound of formula-8 (100 g), acetone (400 ml) and triethyl amine (130 g) at 25-30° C. The reaction mixture was heated to 45° C. and stirred the reaction mixture for 7 hrs 30 min at the same temperature. After completion of the reaction, distilled off the solvent from the reaction mixture and the reaction mixture was cooled to 25-30° C. Water and dichloromethane were added to the reaction mixture and stirred for 15 min. Both the organic and aqueous layers were separated and the aqueous layer was extracted thrice with dichloromethane. Combined the organic layers and distilled off the solvent to get the title compound as solid. Cyclohexane (500 ml) was added to the obtained compound and stirred for 1 hr at 25-30° C. Filtered the solid, washed with cyclohexane and dried to get the title compound.

Yield: 140.0 gm; M.R: 110-115° C.

### Example-2

### Preparation of N-((3R,4R)-1-benzyl-4-methylpiperidin-3-yl)-N-methyl-7-(methylsulfonyl)-7H-pyrrolo[2,3-d]pyrimidin-4-amine (Formula-4a)

4-Chloro-7-(methylsulfonyl)-7H-pyrrolo[2,3-d]pyrimidine compound of formula-3a (13 gm) was added to a mixture of water (65 ml), potassium carbonate (19.3 gm) and (3R,4R)-1-benzyl-N,4-dimethylpiperidin-3-amine compound of formula-2 (12.3 gm) at 25-30° C. Heated the reaction mixture to 90° C. and stirred for 9 hrs at the same temperature. After completion of the reaction, cooled the reaction mixture to 25° C. Extracted the reaction mixture with dichloromethane. Distilled off the solvent completely from the organic layer to get the title compound as residue.

Yield: 18.0 gm.

### Example-3

### Preparation of N-((3R,4R)-1-benzyl-4-methylpiperidin-3-yl)-N-methyl-7-(methylsulfonyl)-7H-pyrrolo[2,3-d]pyrimidin-4-amine (Formula-4a)

4-Chloro-7-(methylsulfonyl)-7H-pyrrolo[2,3-d]pyrimidine compound of formula-3a (5 gm) was added to a mixture of isopropanol (25 ml), sodium bicarbonate (3.6 ml), water (25 ml) and (3R,4R)-1-benzyl-N,4-dimethylpiperidin-3-amine compound of formula-2 (4.7 gm) at 25-30° C. Heated the reaction mixture to 80-85° C. and stirred for 40 hrs at the same temperature. After completion of the reaction, distilled off the solvent from the reaction mixture to obtain residue. Cooled the residue to 20-25° C. and extracted with dichloromethane. Distilled off the solvent completely to get the title compound.

Yield: 8.93 gm.

### Example-4

### Preparation of (3R,4R)-(1-benzyl-4-methyl-piperidin-3yl)-methyl-(7H-pyrrolo[2,3-4:1]pyrimidin-4-yl)amine (Formula-21)

Sodium hydroxide (62 gm) was dissolved in water (60 ml) at 25-30° C. N-((3R,4R)-1-benzyl-4-methylpiperidin-3-yl)-N-methyl-7-(methylsulfonyl)-7H-pynolo[2,3-d]pyrimidin-4-amine compound of formula-4a (17 gm) was added to the above aqueous sodium hydroxide solution at 25-30° C. Heated the reaction mixture to 98100° C. and stirred for 6 hrs at, the same temperature. After completion of the reaction, cooled the reaction mixture to 25-30° C. Extracted the reaction mixture with dichloromethane and distilled off the solvent completely from the organic layer to get the title compound as residue.

Yield: 12.0 gm.

### Example-5

### Preparation of methyl-1-[(3R,4R)-4-methyl-piperidin-3-yl]-(7H-pyrrolo [2,3-d]pyrimidin-4-yl)amine (Formula-6)

Palladium hydroxide (2 gm) was added to a mixture of isopropanol (50 ml), water (50 ml), acetic acid (2.2 ml) and (3R,4R)-(1-Benzyl-4-methyl-piperidin-3yl)-methyl-(7H-pyrrolo[2,3-d]pyrimidin-4-yl)amine compound of formula-21 (10 gm) under nitrogen atmosphere. The reaction mixture was hydrogenated for 6 hrs under a hydrogen gas pressure of 3-4 Kg/cm² at 30-35° C. After completion of the reaction, catalyst was filtered off through hyflow bed. To the obtained filtrate, aqueous sodium hydroxide solution was added and then distilled off the solvent. Cooled the reaction mixture to 25-30° C. and toluene was added to the reaction mixture and stirred for 15 minutes. Both the organic and aqueous layers were separated and aqueous layer was extracted thrice with toluene. Combined the organic layers and distilled off the solvent from the organic layer to get the title compound.

Yield: 5.4 gm.

### Example-6

### Preparation of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate (Formula-1a)

Methyl-1-[(3R,4R)-4-methyl-piperidin-3-yl]-(7H-pyrrolo[2,3-d]pyrimidin-4-yl)amine compound of formula-6 (2 gm) was dissolved in n-butanol (6 ml) at 25-30° C. Ethyl cyanoacetate (1.84 gm) and 1,8-diazabicyclo[5.4.0]undec-7-ene (0.61 gm) were added to the above reaction mixture at 25-30° C. Heated the reaction mixture to 40-45° C. and stirred for 24 hrs at the same temperature. 2-hydroxy-1,2,3-propanetricarboxylic acid (3.4 gm), water (3 ml) and n-butanol (16 ml) were added to the above reaction mixture at 40-45° C. The reaction mixture was heated to. 80-85° C. and stirred for 60 min at the same temperature. After completion of the reaction, cooled the reaction mixture to 20-25° C. and stirred for 2 hrs at the same temperature. Filtered the precipitated solid, washed with n-butanol and dried to get the title compound.

Yield: 3.0 gm.

### Example-7

### Purification of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate (Formula-1a)

(3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate (9 gm) was dissolved in water (590 ml) at 70° C. and stirred for 20 min at the same temperature. Acidic carbon was added to the reaction mixture and stirred at 70-75° C. for 30 min. Filtered the reaction mixture through hyflow bed and the obtained filtrate was cooled to 20-25° C. Acetone (85 ml) was added to the filtrate at 20-25° C. and stirred for 2 hrs at the same temperature. Filtered the precipitated solid, washed with acetone and dried to get the title compound.

Yield: 6.8 gm. Purity by HPLC: 99.74%.

### Example-8

### Purification of (3R,4R)-1-benzyl-N,4-dimethylpiperidin-3-amine (Formula-2)

Conc.HCl (55 ml) was slowly added to a pre-cooled solution of (3R,4R)-1-benzyl-N,4-dimethylpiperidin-3-amine compound of formula-2 (50 gm) in acetone (500 ml) at 0-5° C. and stirred the reaction mixture for 60 min at the same temperature. Filtered the precipitated solid, washed with acetone. The obtained compound was added to ethanol (500 ml) at 25-30° C. Heated the reaction mixture to 75-80° C. and stirred for 45 min at the same temperature. Cooled the reaction mixture to 25-30° C. and stirred for 45 min at the same temperature. Filtered the solid and washed with ethanol. The obtained compound was added to water (150 ml) at 25-30° C. Basified the reaction mixture using aqueous sodium hydroxide solution. Extracted the reaction mixture with toluene and distilled off the solvent under reduced pressure to get the title compound.

Yield: 42.0 gm.

Chiral purity by HPLC: 99.96%; diastereomer 1: 0.04%; Diastereomer 2: Not detected; S,S-isomer: Not detected.

### Example-9

### Preparation of (3R,4R)-(1-benzyl-4-methyl-piperidin-3yl)-methyl-(7H-pyrrolo[2,3-d]pyrimidin-4-yl)amine (Formula-21)

Methanesulfonyl chloride (1.9 Kg) was slowly added to a mixture of 4-chloro-7H-pyrrolo[2,3-d]pyrimidine compound of formula-8 (1.5 Kg), triethylamine (2.3 Kg) and acetone (12 Lt) at 25-30° C. Heated the reaction mixture to 45-50° C. and stirred for 8 hrs at the same temperature. Distilled off the solvent completely from the reaction mixture under reduced pressure. Cooled the obtained compound to 25-30° C., water and dichloromethane were added and stirred for 15 min at the same temperature. Both the organic and aqueous layers were separated and washed the organic layer with aqueous sodium bicarbonate solution. Distilled off the solvent completely from the organic layer under reduced pressure and co-distilled with acetonitrile. Cooled the obtained compound to 25-30° C., acetonitrile (7.5 Lt) was added and stirred for 15 min at the same temperature. The obtained reaction mixture was added to a mixture of acetonitrile (7.5 Lt) and sodium bicarbonate (1.2 Kg) at 25-30° C. (3R,4R)-1-benzyl-N,4-dimethylpiperidin-3-amine compound of formula-2 (2.13 Kg) was added to the reaction mixture. Heated the reaction mixture to 80-85° C. and stirred for 47 hrs at the same temperature. Cooled the reaction mixture to 60-65° C. and distilled off the solvent completely. Cooled the obtained compound to 25-30° C., water and toluene were added and stirred the reaction mixture for 15 min at the same temperature. Both the organic and aqueous layers were separated and the aqueous layer was extracted with toluene. Combined the organic layers and aqueous sodium hydroxide solution (15.3 Kg of sodium hydroxide in 12 Lt of water) was added at 25-30° C. Heated the reaction mixture to 85-90° C. and stirred for 5 hrs at the same temperature. Cooled the reaction mixture to 25-30° C. and filtered. Both the organic and aqueous layers were separated and water was added to the organic layer. Adjusted the pH of the reaction mixture to 8.5 using acetic acid. Both the organic and aqueous layers were separated. Distilled off the solvent completely from the organic layer under reduced pressure and co-distilled with cyclohexane. 39 Lt of cyclohexane was added to the obtained compound at 25-30° C. Heated the reaction mixture to 60-65° C. Slowly cooled the reaction mixture to 15-20° C. and stirred for 1 hr at the same temperature. Filtered the solid, washed with cyclohexane and dried to get the title compound.

Yield: 2.2 Kg.

### Example-10

### Preparation of (3R,4R)-(1-benzyl-4-methyl-piperidin-3yl)-methyl-(7H-pyrrolo[2,3-d]pyrimidin-4-yl)amine (Formula-21)

The title compound is prepared according to the process disclosed in example-9 by using p-toluenesulfonyl chloride instead of methanesulfonyl chloride.

Yield: 2.0 Kg.

### Example-11

### Preparation of methyl-1-[(3R,4R)-4-methyl-piperidin-3-yl]-(7H-pyrrolo[2,3-d]pyrimidin-4-yl)amine (Formula-6)

A slurry of palladium hydroxide (0.3 Kg) in water (10 Lt) was slowly added to a mixture of (3R,4R)-(1-benzyl-4-methyl-piperidin-3yl)-methyl-(7H-pyrrolo[2,3-d]pyrimidin-4-yl)amine compound of formula-21 (2 Kg), isopropyl alcohol (30 Lt), water (10 Lt) and acetic acid (0.4 Lt) at 25-30° C. under nitrogen atmosphere. 3-4 Kg/cm² of hydrogen gas pressure was applied to the reaction mixture at 25-30° C. and stirred for 8 hrs under the same conditions. Slowly released hydrogen gas pressure and flushed the reaction mixture with nitrogen. Filtered the reaction mixture and basified the filtrate using aqueous sodium hydroxide solution. Distilled off isopropyl alcohol completely from the reaction mixture under reduced pressure. Dichloromethane was added to the reaction mixture at 25-30° C. and stirred for 15 min at the same temperature. Both the organic and aqueous layers were separated and the aqueous layer was extracted with dichloromethane. Combined the organic layers and distilled off the solvent completely under reduced, pressure and co-distilled with cyclohexane. 20 Lt of cyclohexane was added to the obtained compound at 25-30° C. Heated the reaction mixture to 50-55° C. and stirred for 30 min at the same temperature. Cooled the reaction mixture to 25-30° C. and stirred for 45 min at the same temperature. Filtered the solid, washed with cyclohexane and dried to get the title compound.

Yield: 1.24 Kg; Dihydro impurity: 0.03%.

### Example-12

### Preparation of methyl-1-[(3R,4R)-4-methyl-piperidin-3-yl]-(7H-pyrrolo[2,3-d]pyrimidin-4-yl)amine (Formula-6)

A slurry of palladium hydroxide (0.3 Kg) in water (10 Lt) was slowly added to a mixture of (3R,4R)-(1-benzyl-4-methyl-piperidin-3yl)-methyl-(7H-pyrrolo[2,3-d]pyrimidin-4-yl)amine compound of formula-21 (2 Kg), isopropyl alcohol (30 Lt), water (10 Lt) and acetic acid (0.4 Lt) at 40-45° C. under nitrogen atmosphere. 3-4 Kg/cm² of hydrogen gas pressure was applied to the reaction mixture at 40-45° C. and stirred for 8 hrs under the same conditions. Slowly released hydrogen gas pressure and flushed the reaction mixture with nitrogen. Filtered the reaction mixture and basified the filtrate using aqueous sodium hydroxide solution. Distilled off isopropyl alcohol completely from the reaction mixture under reduced pressure. Dichloromethane was added to the reaction mixture at 25-30° C. and stirred for 15 min at the same temperature. Both the organic and aqueous layers were separated and the aqueous layer was extracted with dichloromethane. Combined the organic layers and distilled off the solvent completely under reduced pressure and co-distilled with cyclohexane. 20 Lt of cyclohexane was added to the obtained compound at 25-30° C. Heated the reaction mixture to 50-55° C. and stirred for 30 min at the same temperature. Cooled the reaction mixture to 25-30° C. and stirred for 45 min at the same temperature. Filtered the solid, washed with cyclohexane and dried to get the title compound.

Yield: 1.15 Kg; Dihydro impurity: 0.3%.

### Example-13

### Preparation of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate (Formula-1a)

A mixture of methyl-1-[(3R,4R)-4-methyl-piperidin-3-yl]-(7H-pyrrolo[2,3-d]pyrimidin-4-yl)amine compound of formula-6 obtained in example-12 (1.2 Kg) and n-butanol (6 Lt) was stirred for 15 min at 25-30° C. Ethyl cyanoacetate (1.6 Kg), 1,8-diazabicyclo[5.4.0]undec-7-ene (0.5 Kg) were added to the reaction mixture at 25-30° C. Heated the reaction mixture to 55-60° C. and stirred for 23 hrs at the same temperature. Cooled the reaction mixture to 25-30° C. and a solution of 2-hydroxy-1,2,3-propanetricarboxylic acid (2 Kg) in water (1.8 Lt) was added. n-butanol (7.2 Lt) was added to the reaction mixture, heated to 80-85° C. and stirred for 60 min at the same temperature. Cooled the reaction mixture to 25-30° C. and stirred for 2 hrs at the same temperature. Filtered the precipitated solid, washed with n-butanol and dried to get the title compound.

Yield: 2.08 Kg; Dihydro impurity: 0.3%.

### Example-14

### Preparation of (3R,4R)-4-methyl-3-(methyl-7H-pyrrolo[2,3-d]pyrimidin-4-ylamino)-β-oxo-1-piperidine propanenitrile (Formula-1)

A mixture of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate compound of formula-1a obtained in example-13 (600 gm), dichloromethane (3 Lt) and water (3 Lt) was stirred for 10 min at 25-30° C. Cooled the reaction mixture to 15-20° C. and stirred for 10 min at the same temperature. 20% aqueous sodium carbonate solution was added to the reaction mixture and stirred for 6 hrs. Filtered the solid, washed with dichloromethane followed by water and then dried to get the title compound.

Yield: 360.0 gm. Dihydro impurity: 0.03%.

### Example-15

### Preparation of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate (Formula-1a)

(3R,4R)-4-methyl-3-(methyl-7H-pyrrolo[2,3-d]pyrimidin-4-ylamino)-β-oxo-1-piperidine propanenitrile compound of formula-1 obtained in example-14 (15.5 gm) was added to a solution of 2-hydroxy-1,2,3-propanetricarboxylic acid (11.5 gm) in water (800 ml) at 70-75° C. Heated the reaction mixture to 85-90° C. and stirred for 1 hr at the same temperature. Charcoal (2 gm) was added to the reaction mixture and stirred for 15 min at the same temperature. Filtered the reaction mixture through hyflow bed and washed with water. Acetone (450 ml) was added to the filtrate at 25-30° C. Cooled the reaction mixture to 0-5° C. and stirred for 4 hrs at the same temperature. Filtered the solid, washed with acetone and dried the material to get the title compound.

Yield: 19.0 gm; Dihydro impurity: 0.02%.

### Example-16

### Preparation of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate (Formula-1a)

A mixture of methyl-1-[(3R,4R)-4-methyl-piperidin-3-yl]-(7H-pyrrolo[2,3-d]pyrimidin-4-yl)amine compound of formula-6 obtained in example-11 (1.2 Kg) and n-butanol (6 Lt) was stirred for 15 min at 25-30° C. Ethyl cyanoacetate (1.6 Kg), 1,8-diazabicyclo[5.4.0]undec-7-ene (0.5 Kg) were added to the reaction mixture at 25-30° C. Heated the reaction mixture to 55-60° C. and stirred for 23 hrs at the same temperature. Cooled the reaction mixture to 25-30° C. and a solution of 2-hydroxy-1,2,3-propanetricarboxylic acid (2 Kg) in water (1.8 Lt) was added. n-butanol (7.2 Lt) was added to the reaction mixture, heated to 80-85° C. and stirred for 60 min at the same temperature. Cooled the reaction mixture to 25-30° C. and stirred for 2 hrs at the same temperature. Filtered the precipitated solid, washed with n-butanol and dried to get the title compound. Yield: 2.14 Kg; Dihydro impurity: 0.03%.

### Example-17

### Purification of Compound of Formula-1a

A mixture of (3R,4R)-4-methyl-3-(methyl-7H-pyrrolo[2,3-d]pyrimidin-4-ylamino)-β-oxo-1-piperidine propanenitrile 2-hydroxy-1,2,3-propanetricarboxylate compound of formula-1a (2 Kg) and water (60 Lt) was heated to 90-95° C. and stirred for 90 min at the same temperature. Reduced the temperature of the reaction mixture to 50-55° C. and charcoal (0.2 kg) was added. Heated the reaction mixture to 90-95° C. and stirred for 30 min at the same temperature. Filtered the reaction mixture through hyflow bed and hot water (4 Lt) was added to the filtrate. Cooled the filtrate to 25-30° C. and acetone (40 Lt) was added. Further cooled the reaction mixture to 0-5° C. and stirred for 6 hrs at the same temperature. Filtered the precipitated solid, washed with chilled acetone and dried to get the title compound. The PXRD pattern of the obtained compound is matches with the PXRD pattern of crystalline form disclosed in U.S. Pat. No. 6,965,027B2.

Yield: 1.5 Kg; Purity by HPLC: 99.71%.

Amine impurity: 0.08%; Dihydro impurity: 0.02%; Benzyl impurity: Not detected; Diastereomer 1: Not detected; Diastereomer 2: 0.02%; S,S-isomer: 0.03%.

Particle size distribution (before micronization): D(0.1) is 1.83 gm, D(0.5) is 7.68 gm, D(0.9) is 29.58 gm;

Particle size distribution (after micronization): D(0.1) is 0.83 gm, D(0.5) is 2.20 gm, D(0.9) is 4.27 gm.

1-19. (canceled)

20. A process for the preparation of (3R,4R)-4-methyl-3-(methyl-7H-pyrrolo[2,3-d]pyrimidin-4-ylamino)-β-oxo-1-piperidine propanenitrile compound of formula-1 and its citrate salt compound of formula-1a, comprising:
a) reacting the 4-chloro-7H-pyrrolo[2,3-d]pyrimidine compound of formula-8 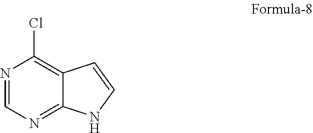
with methanesulfonyl chloride in the presence of a suitable base in a suitable solvent to provide 4-chloro-7-(methylsulfonyl)-7H-pyrrolo[2,3-d]pyrimidine of formula-3a, 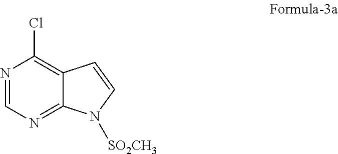 [SMILES: CS(=O)(=O)n1ccc2c(Cl)ncnc21]
b) reacting the compound of formula-3a with (3R,4R)-1-benzyl-N,4-dimethylpiperidin-3-amine compound of formula-2 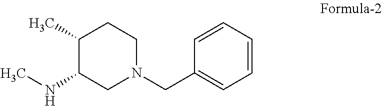 [SMILES: CN[C@H]1CN(Cc2ccccc2)CC[C@H]1C]
in the presence of a base in a solvent to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a, 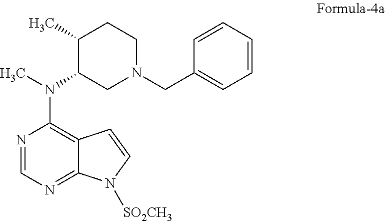 [SMILES: C[C@@H]1CCN(Cc2ccccc2)C[C@@H]1N(C)c1ncnc2c1ccn2S(C)(=O)=O]
c) deprotecting the compound of formula-4a by treating it with a base in a solvent to provide N-((3R,4R)-1-benzyl-4-methylpiperidin-3-yl)-N-methyl-7H-pyrrolo[2,3-d]pyrimidin-4-amine compound of formula-21, 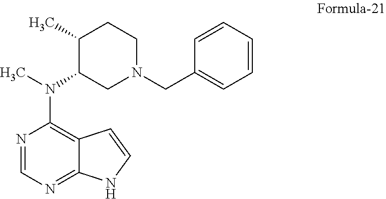 [SMILES: C[C@@H]1CCN(Cc2ccccc2)C[C@@H]1N(C)c1ncnc2[nH]ccc12]
d) treating the compound of formula-21 with a debenzylating agent in a solvent to provide N-methyl-N-((3R,4R)-4-methylpiperidin-3-yl)-7H-pyrrolo[2,3-d]pyrimidin-4-amine compound of formula-6, 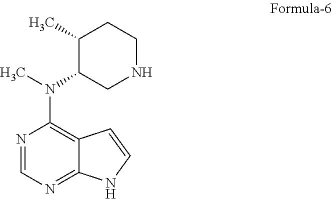 [SMILES: C[C@@H]1CCNC[C@@H]1N(C)c1ncnc2[nH]ccc12]
e) reacting the compound of formula-6 with 2-cyanoacetyl derivative compound of general formula-(a) 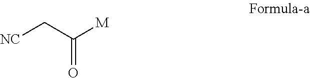
wherein, ‘M’ represents ‘X’ or ‘OR’; ‘X’ is Cl, Br; ‘R’ is C₁-C₆ alkyl;
in the presence of a base in a solvent to provide compound of formula-1, and
f) treating the compound of formula-1 with citric acid in a solvent to provide compound of formula-1a.

21. The process according to claim 20, wherein, in step-(a), step-(b) & step-(e) the base is selected from organic bases, inorganic bases or their mixtures; in step-(c) the base is selected from alkali metal hydroxides, alkali metal alkoxides; in step-(d) the debenzylating agent is selected from concentrated HCl, Pd, Pd/C, Pd(OH)₂/C, palladium acetate, Raney Ni, Pt/C, platinum oxide, platinum black, Rh/C, Ru, and Ir optionally in combination with hydrogen; in step-(a) to step-(f) the solvent is selected from ether solvents, ester solvents, chloro solvents, hydrocarbon solvents, polar solvents, polar-aprotic solvents, ketone solvents, alcohol solvents, nitrile solvents, acetic acid, formic acid or their mixtures.

22. The process according to claim 20, wherein, in step-d) the debenzylation is carried out at a temperature ranging from 0-40° C., preferably at 20-30° C.

23. A process for the preparation of (3R,4R)-4-methyl-3-(methyl-7H-pyrrolo[2,3-d]pyrimidin-4-ylamino)-β-oxo-1-piperidine propanenitrile compound of formula-1 and its citrate salt compound of formula-1a, comprising:
a) reacting the 4-chloro-7H-pyrrolo[2,3-d]pyrimidine compound of formula-8 with methanesulphonyl chloride in the presence of triethyl amine in acetone to provide 4-chloro-7-(methylsulfonyl)-7H-pyrrolo[2,3-d]pyrimidine compound of formula-3a,
b) reacting the compound of formula-3a with (3R,4R)-1-benzyl-N,4-dimethylpiperidin-3-amine compound of formula-2 in the presence of sodium bicarbonate in acetonitrile to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a,
c) deprotecting the compound of formula-4a by treating it with aqueous sodium hydroxide in toluene to provide N-((3R,4R)-1-benzyl-4-methylpiperidin-3-yl)-N-methyl-7H-pyrrolo[2,3-d]pyrimidin-4-amine compound of formula-21,
d) treating the compound of formula-21 with palladium hydroxide in aqueous isopropyl alcohol and in presence of catalytic amount of acetic acid to provide N-methyl-N-((3R,4R)-4-methylpiperidin-3-yl)-7H-pyrrolo[2,3-d]pyrimidin-4-amine compound of formula-6,
e) reacting the compound of formula-6 with ethyl cyanoacetate compound of formula-al 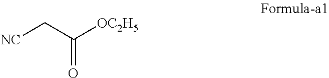
in presence of 1,8-diazabicyclo[5.4.0]undec-7-ene in n-butanol to provide a compound of formula-1, and
f) treating the compound of formula-1 with citric acid in aqueous n-butanol to provide compound of formula-1a.

24. A compound having the structural formula;

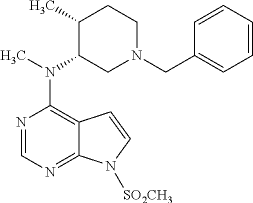 [SMILES: C[C@@H]1CCN(Cc2ccccc2)C[C@@H]1N(C)c1ncnc2c1ccn2S(C)(=O)=O]

25. The process according to claim 20, wherein, the preparation of N-((3R,4R)-1-benzyl-4-methylpiperidin-3-yl)-N-methyl-7-(methylsulfonyl)-7H-pyrrolo[2,3-d]pyrimidin-4-amine compound of formula-4a, comprises reacting the 4-chloro-7-(methylsulfonyl)-7H-pyrrolo[2,3-d]pyrimidine compound of formula-3a with (3R,4R)-1-benzyl-N,4-dimethylpiperidin-3-amine compound of formula-2 in the presence of a base in a solvent to provide compound of formula-4a.

26. The process according to claim 25, wherein, the base is selected from organic bases, inorganic bases or their mixtures; and the solvent is selected from nitrile solvents, alcohol solvents, polar solvents, ether solvents, ester solvents, hydrocarbon solvents, polar-aprotic solvents, ketone solvents, chloro solvents or their mixtures.

27. The process according to claim 25, wherein, the preparation of N-((3R,4R)-1-benzyl-4-methylpiperidin-3-yl)-N-methyl-7-(methylsulfonyl)-7H-pyrrolo[2,3-d]pyrimidin-4-amine compound of formula-4a, comprises reacting the 4-chloro-7-(methylsulfonyl)-7H-pyrrolo[2,3-d]pyrimidine compound of formula-3a with (3R,4R)-1-benzyl-N,4-dimethylpiperidin-3-amine compound of formula-2 in the presence of sodium bicarbonate in acetonitrile to provide compound of formula-4a.

28. The process according to claim 20, wherein, the preparation of N-((3R,4R)-1-benzyl-4-methylpiperidin-3-yl)-N-methyl-7H-pyrrolo[2,3-d]pyrimidin-4-amine compound of formula-21, comprising of deprotecting the N-((3R,4R)-1-benzyl-4-methylpiperidin-3-yl)-N-methyl-7-(methylsulfonyl)-7H-pyrrolo[2,3-d]pyrimidin-4-amine compound of formula-4a by treating it with a base in a solvent to provide compound of formula-21.

29. The process according to claim 28, wherein, the base is selected from alkali metal hydroxides, alkali metal alkoxides; and the solvent is selected from nitrile solvents, alcohol solvents, polar solvents, ether solvents, ester solvents, hydrocarbon solvents, polar-aprotic solvents, ketone solvents, chloro solvents or their mixtures.

30. The process according to claim 28, wherein, the preparation of N-((3R,4R)-1-benzyl-4-methylpiperidin-3-yl)-N-methyl-7H-pyrrolo[2,3-d]pyrimidin-4-amine compound of formula-21, comprises deprotecting the N-((3R,4R)-1-benzyl-4-methylpiperidin-3-yl)-N-methyl-7-(methylsulfonyl)-7H-pyrrolo[2,3-d]pyrimidin-4-amine compound of formula-4a by treating it with aqueous sodium hydroxide solution in toluene to provide a compound of formula-21.

31. The process according to claim 20, wherein, the preparation of (3R,4R)-1-benzyl-N,4-dimethylpiperidin-3-amine compound of formula-2 having less than 0.1% of diastereomer impurity, comprising:
a) treating the compound of formula-2 with an acid in a solvent to provide a corresponding acid-addition salt,
b) optionally slurrying the acid-addition salt obtained in step-a) in a solvent at a suitable temperature, and
c) treating the reaction mixture with a base to provide a compound of formula-2 having less than 0.1% of diastereomer impurity.

32. The process according to claim 31, wherein, in step-a) the acid is selected from inorganic acids or organic acids; in step-b) the temperature ranges from 25-30° C. to a reflux temperature of the solvent used; in step-c) the base is selected from inorganic bases; and in step-a) & step-b) the solvent is selected from nitrile solvents, alcohol solvents, polar solvents, ether solvents, ester solvents, hydrocarbon solvents, polar-aprotic solvent, ketone solvents, chloro solvents or their mixtures.

33. The process according to claim 31, wherein, the preparation of (3R,4R)-1-benzyl-N,4-dimethylpiperidin-3-amine compound of formula-2 having less than 0.1% of diastereomer impurity, comprises:
a) treating the compound of formula-2 with concentrated HCl in acetone,
b) filtering the hydrochloride salt of compound of formula-2,
c) adding the hydrochloride salt obtained in step-b) to ethanol to form a reaction mixture,
d) heating the reaction mixture,
e) cooling the reaction mixture, and
f) neutralizing the reaction mixture with aqueous sodium hydroxide solution to provide a compound of formula-2 having less than 0.1% of diastereomer impurity.

34. Use of 4-chloro-7-(methylsulfonyl)-7H-pyrrolo[2,3-d]pyrimidine and N-((3R,4R)-1-benzyl-4-methylpiperidin-3-yl)-N-methyl-7-(methylsulfonyl)-7H-pyrrolo[2,3-d]pyrimidin-4-amine obtained according to claim-20 in the preparation of (3R,4R)-4-methyl-3-(methyl-7H-pyrrolo[2,3-d]pyrimidin-4-ylamino)-β-oxo-1-piperidine propanenitrile or its salts.

35. (3R,4R)-4-methyl-3-(methyl-7H-pyrrolo[2,3-d]pyrimidin-4-ylamino)-β-oxo-1-piperidine propanenitrile or its citrate salt obtained according to claim-20 having a dihydro impurity less than 0.15% by HPLC, preferably less than 0.05% by HPLC.

36. (3R,4R)-4-methyl-3-(methyl-7H-pyrrolo[2,3-d]pyrimidin-4-ylamino)-β-oxo-1-piperidine propanenitrile citrate obtained according to claim-20 having a particle size distribution of D₉₀ less than or equal to 200 μm.

37. (3R,4R)-4-methyl-3-(methyl-7H-pyrrolo[2,3-d]pyrimidin-4-ylamino)-β-oxo-1-piperidine propanenitrile citrate obtained according to claim-20 having a particle size distribution of D₉₀ less than or equal to 100 μm, preferably less than or equal to 50 μm.

38. The process according to claim-20, wherein, the preparation of N-((3R,4R)-1-benzyl-4-methylpiperidin-3-yl)-N-methyl-7H-pyrrolo[2,3-d]pyrimidin-4-amine compound of formula-21, comprises:
a) reacting the 4-chloro-7H-pyrrolo[2,3-d]pyrimidine compound of formula-8 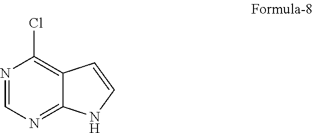
with methanesulfonyl chloride in the presence of a base in a solvent to provide 4-chloro-7-(methylsulfonyl)-7H-pyrrolo[2,3-d]pyrimidine of formula-3a, 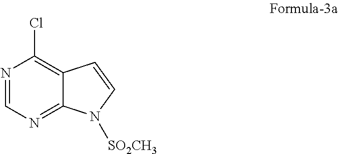 [SMILES: CS(=O)(=O)n1ccc2c(Cl)ncnc21]
b) reacting the compound of formula-3a in-situ with (3R,4R)-1-benzyl-N,4-dimethylpiperidin-3-amine compound of formula-2 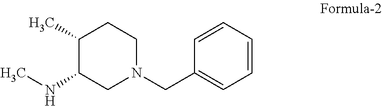 [SMILES: CN[C@H]1CN(Cc2ccccc2)CC[C@H]1C]
in the presence of a base in a solvent to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a, 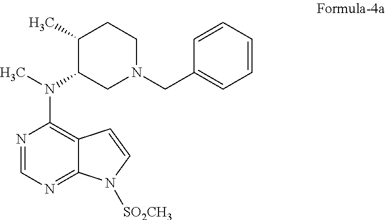 [SMILES: C[C@@H]1CCN(Cc2ccccc2)C[C@@H]1N(C)c1ncnc2c1ccn2S(C)(=O)=O]
c) deprotecting the compound of formula-4a by treating in-situ with a base in a solvent to provide N-((3R,4R)-1-benzyl-4-methylpiperidin-3-yl)-N-methyl-7H-pyrrolo[2,3-d]pyrimidin-4-amine compound of formula-21, 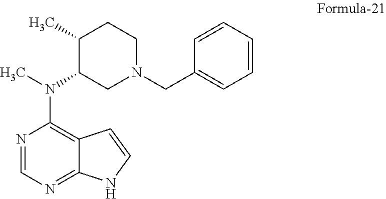 [SMILES: C[C@@H]1CCN(Cc2ccccc2)C[C@@H]1N(C)c1ncnc2[nH]ccc12]
d) optionally, treating the compound of formula-21 with an alcoholic base to provide pure N-((3R,4R)-1-benzyl-4-methylpiperidin-3-yl)-N-methyl-7H-pyrrolo[2,3-d]pyrimidin-4-amine compound of formula-21.

39. The process according to claim-38, wherein, the preparation of N-((3R,4R)-1-benzyl-4-methylpiperidin-3-yl)-N-methyl-7H-pyrrolo[2,3-d]pyrimidin-4-amine compound of formula-21, comprises:
a) reacting the 4-chloro-7H-pyrrolo[2,3-d]pyrimidine compound of formula-8 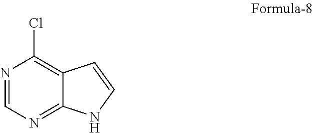
with methanesulfonyl chloride in the presence of triethyl amine in acetone to provide 4-chloro-7-(methylsulfonyl)-7H-pyrrolo[2,3-d]pyrimidine of formula-3a, 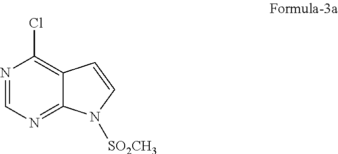 [SMILES: CS(=O)(=O)n1ccc2c(Cl)ncnc21]
b) reacting the compound of formula-3a in-situ with (3R,4R)-1-benzyl-N,4-dimethylpiperidin-3-amine compound of formula-2 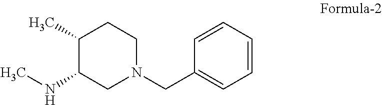 [SMILES: CN[C@H]1CN(Cc2ccccc2)CC[C@H]1C]
in the presence of sodium bicarbonate in acetonitrile to provide N-((3R,4R)-1-benzyl-4-methylpiperidin-3-yl)-N-methyl-7-(methylsulfonyl)-7H-pyrrolo[2,3-d]pyrimidin-4-amine compound of formula-4a, 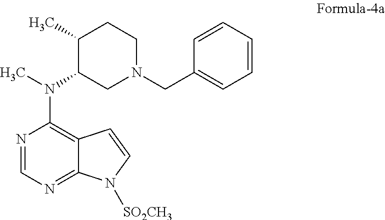 [SMILES: C[C@@H]1CCN(Cc2ccccc2)C[C@@H]1N(C)c1ncnc2c1ccn2S(C)(=O)=O]
c) deprotecting the compound of formula-4a by treating in-situ with aqueous sodium hydroxide in toluene to provide N-((3R,4R)-1-benzyl-4-methylpiperidin-3-yl)-N-methyl-7H-pyrrolo[2,3-d]pyrimidin-4-amine compound of formula-21, and 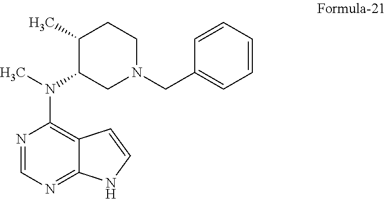 [SMILES: C[C@@H]1CCN(Cc2ccccc2)C[C@@H]1N(C)c1ncnc2[nH]ccc12]
d) treating the compound of formula-21 with methanolic potassium hydroxide to provide pure N-((3R,4R)-1-benzyl-4-methylpiperidin-3-yl)-N-methyl-7H-pyrrolo[2,3-d]pyrimidin-4-amine compound of formula-21.
